(19) United States
(12) Patent Application Publication
(10) Pub. No.: US 2006/0127953 A1
(43) Pub. Date: Jun. 15, 2006

(54) Method of diagnosing osteolysis

(75) Inventors: Sean Frost, New Hampshire (CA); Steven McDonald, London (CA); Kelly Summers, London (CA)

Correspondence Address: FOLEY AND LARDNER LLP;SUITE 500, 3000 K STREET NW, WASHINGTON, DC, 20007, US

(73) Assignee: London Health Sciences Centre Research Inc., London, Ontario (CA)

(21) Appl. No.: 10/528,339

(22) PCT Filed: Sep. 18, 2003
(86) PCT No.: PCT/CA03/01360

§ 371 Date: Dec. 29, 2005

Related U.S. Application Data
(60) Provisional application No. 60/411,351, filed on Sep. 18, 2002.

(51) Int. Cl. G01N 33/567; G01N 33/53; G01N 33/574

(52) U.S. Cl. 435/7.2; 435/7.23

(57) ABSTRACT

Methods for detecting osteolysis are described. The diagnostic method involves detecting the presence of regulatory T cells in a sample from a patient.

### FIELD OF THE INVENTION

The present invention relates to methods for diagnosing osteolysis.

### BACKGROUND OF THE INVENTION

Osteolysis, and subsequent aseptic loosening, remains one of the most common and devastating complications following total joint arthroplasty. The pathophysiology of osteolysis involves immune cells that are clearing intra-articular debris (wear particles) from the normal wear of material interfaces. Osteolysis occurs after stimulation and differentiation of osteoclasts (bone resorbing cells), and inhibition of osteoblasts (bone forming cells) by cytokines, prostaglandins, and matrix metalloproteinases which are produced primarily by macrophages in response to phagocytosis of submicron wear particles. While evidence suggests that osteolysis results from an increase in osteoclast activity, it appears that an immune response involving activated macrophages triggers the cascade of events. The mechanism of macrophage—osteoclast signalling has not been fully elucidated. While others have described T cell interactions with these macrophages, to date, no authors have investigated the role of CD3⁺CD4⁺CD25⁺ regulatory T cells (T_{REG}) coordinating with macrophages in the osteolytic process. Evidence for involvement of regulatory T cells would contribute to our understanding of this complex biologic response to artificial wear particles in the hip joint, and perhaps offer therapeutic intervention in the future.

### SUMMARY OF THE INVENTION

The present inventors have demonstrated that CD3⁺CD4⁺CD25⁺ regulatory T cells are upregulated in total hip replacement patients with early osteolysis.

Accordingly, the present invention provides a method of detecting osteolysis in a patient comprising:

(a) obtaining a sample containing lymphocytes from the patient; and

(b) determining the presence of regulatory T cells in the sample wherein an increase in regulatory T cells as compared to a control indicates that the patient has osteolysis.

The present invention also includes a kit for detecting osteolysis comprising the necessary reagents to detect regulatory T cells along with instructions for the use thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a graph showing regulatory T cells in loose total hip replacements in synovial tissue, interface tissue and peripheral blood.

FIG. 2 is a graph showing regulatory T cells in peripheral blood from patients with hip replacement as compared to controls.

FIG. 3 is a graph showing regulatory T cells in peripheral blood from patients with hip replacement, osteolysis and pre-failure osteolysis as compared to a control.

FIG. 4 is a graph showing the levels of naïve and memory T cells in various samples.

FIG. 5 is a graph showing the levels of activated T cells in various samples.

### DETAILED DESCRIPTION OF THE INVENTION

As hereinbefore mentioned, the present inventors have demonstrated that CD3⁺CD4⁺CD25⁺ regulatory T cells are upregulated in total hip replacement patients with early osteolysis.

Accordingly, the present invention provides a method of detecting osteolysis in a patient comprising:

(a) obtaining a sample containing lymphocytes from the patient; and

(b) determining the amount of CD4⁺CD25⁺ regulatory T cells in the sample wherein an increase in regulatory T cells as compared to a control indicates that the patient has osteolysis.

The term “regulatory T cell” as used herein means a T lymphocyte having the phenotype or cell surface markers CD4⁺CD25⁺. The cells will also be CD3⁺ as they are T lymphocytes.

The sample can be any sample from a patient containing lymphocytes including, but not limited to, peripheral blood, synovial fluid, synovial tissue and interface tissue between the failed component and bone defect. Preferably, the sample is peripheral blood which is generally the easiest to obtain from a patient.

The presence of T regulatory cells can be assessed by analyzing the sample for T lymphocytes having both of the cell surface antigens CD4 and CD25. The presence of these antigens can be detected using a variety of standard methods known in the art including, but not limited to, fluorescent-tagged antibody cell sorting (FACS), immunochemistry and enzyme linked immunosorbent assay (ELISA). In a preferred embodiment, the presence of the CD4⁺CD25⁺ cells can be determined using two colour FACS wherein each antibody is labelled with a different colour fluorochrome. For example, the anti-CD4 antibody can be labelled with FITC that fluoresces green and the anti-CD25 antibody can be labelled with PE that fluoresces red. Cells that contain both CD4⁺ and CD25⁺ (i.e. regulatory T cells) will fluoresce yellow.

The method of the invention can be used to detect osteolysis resulting from a variety of causes including, but not limited to, total hip replacement, primary metastatic bone cancer and metabolic bone diseases such as Paget disease.

The control can be (1) a sample from a patient that does not have osteolysis that undergoes the same process as the test sample or (2) standard values of regulatory T cells that are known to be present in a person without osteolysis in the same sample type as the test sample. For example, when the patient sample is peripheral blood, the levels of regulatory T cells can be compared to known levels of regulatory T cells in the peripheral blood of people without osteolysis. In this regard, the inventors have determined that normal controls generally have a level of regulatory T cells of 44% (of the total CD4⁺ lymphocytes) in a sample of peripheral blood. Patients with osteolysis have levels of regulatory T cells of about 74% in peripheral blood and about 62% in synovial tissue or interface tissue. Therefore, levels of regulatory T cells of greater than 45%, preferably greater than 50%, more preferably greater than 60% and most preferably greater than 70% (for peripheral blood samples), are indicative of osteolysis.

Accordingly, in a specific embodiment, the present invention provides a method of detecting osteolysis in a patient comprising:

(a) obtaining a sample containing lymphocytes from the patient; and

(b) determining the amount of CD4⁺CD25⁺ regulatory T cells as a percentage of the total CD4⁺ lymphocytes in the sample, wherein a level of regulatory T cells of greater than 45% indicates that the patient has osteolysis.

To measure the % T regulatory cells as a function of total CD4⁺ T cells, lymphocytes are removed from the patient's sample and antibodies that bind to CD4 and CD25 are incubated with the lymphocytes. The sample is assessed for both CD4⁺CD25⁺ cells as well as total CD4⁺ cells and the % CD4⁺CD25⁺ is calculated as a percentage of total CD4⁺ cells. The method used is preferably FACS wherein the antibodies that bind CD4 or CD25 are labelled with a fluorochrome as described above.

The inventors have also determined that the regulatory T cells demonstrate a naïve (CD45RA⁺) profile and are non-activiated (CD80⁻/CD86⁻) in peripheral blood while they are memory-like (CD45RO⁺) and activated (CD80⁺/CD86⁺) at the site of action of osteolysis (e.g. synovial tissue, interface tissue). Consequently, the diagnostic method can also include detecting the presence of CD45RA, CD45RO, CD80 and/or CD86 in the sample from the patient.

The present invention also include a kit for detecting osteolysis which comprise the necessary reagents for detecting CD4⁺CD25⁺ regulatory T cells as well as instructions for the use of the kit. Reagents for detecting the regulatory T cells include antibodies that bind to the surface antigens CD3, CD4 and CD25.

The following non-limiting examples are illustrative of the present invention:

### EXAMPLE

### Example 1

The Role of Regulatory T Cells in Periprosthetic Osteolysis following Primary Total Hip Arthroplasty

Methods: 15 consecutive patients booked for revision total hip arthroplasty secondary to osteolysis/aseptic loosening were included. Intra-operative tissue samples were collected including peripheral blood (PB), synovial fluid (SF), synovial tissue (ST), and interface tissue (IT) between the failed component and the bone defect. Total lymphocytes were isolated from the 4 tissues in vitro, and then analyzed using fluorescent-tagged antibody cell sorting (FACS) (using antibodies that bind to CD4 and antibodies that bind to CD25) for the presence and activation of T_{REG} cells. Samples of ST and IT were frozen and subsequently cut for H & E staining for lymphocytes, and immunohistochemistry for T_{REG} cells. Ten healthy patients and 5 patients with total hip replacements with no evidence of osteolysis were used as controls.

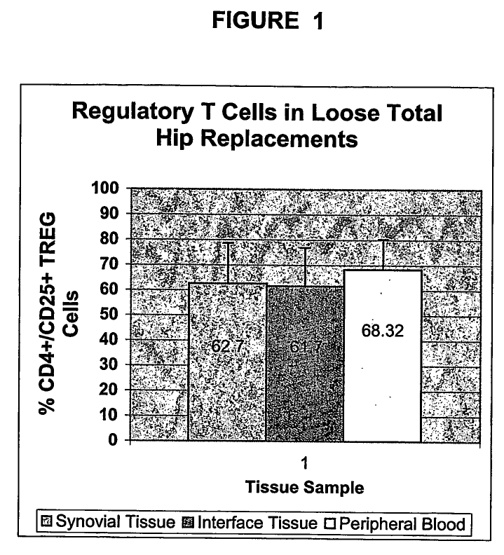

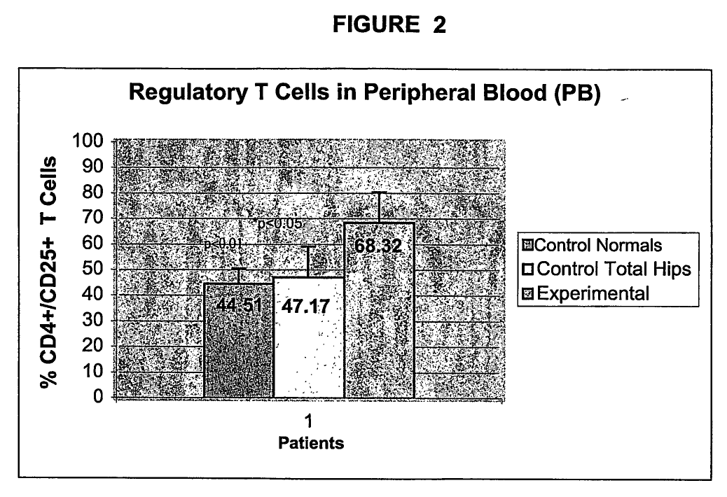

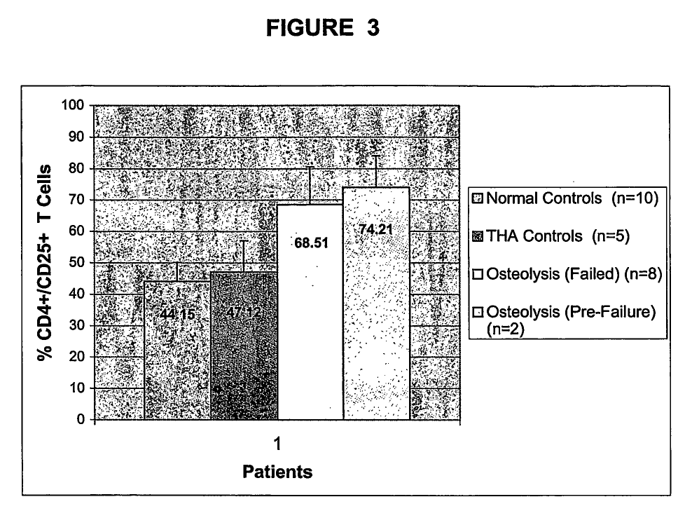

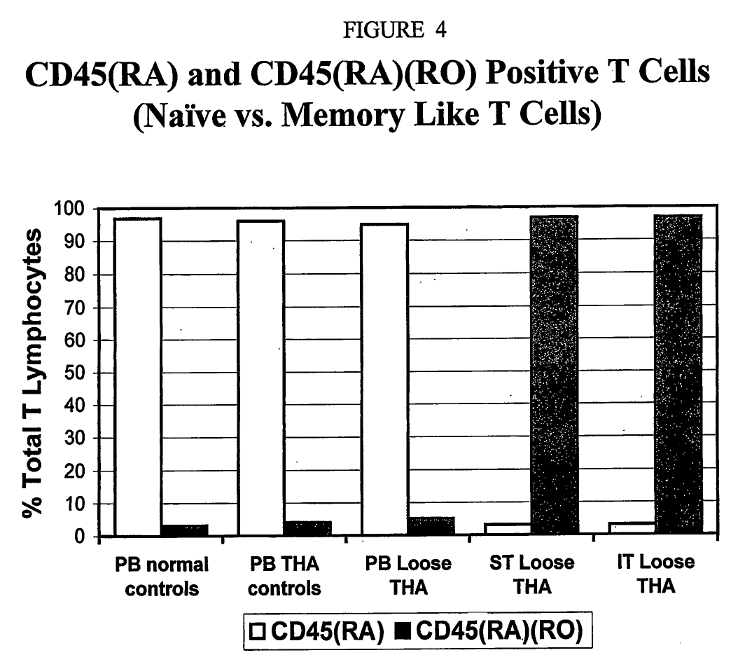

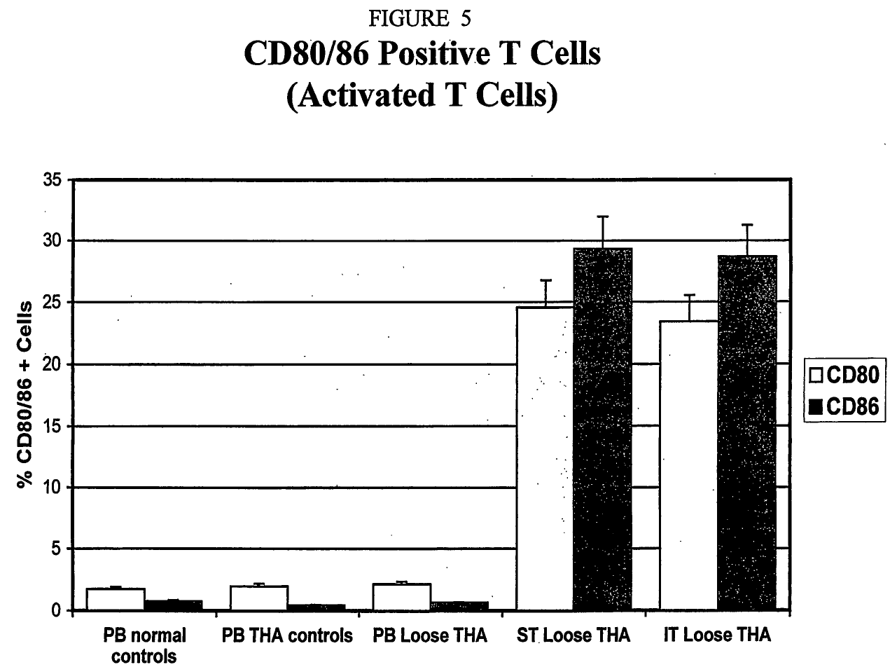

Results: T_{REG} cells were significantly upregulated in the PB (68%) of revision hip patients compared to normal controls PB (44%) (p<0.01) and to patients with total hip replacements with no osteolysis PB (47%) (p<0.05) (see FIG. 2). T_{REG} cells in total hip replacement patients with early osteolysis (as noted on X-ray) PB (74%) were also significantly upregulated when compared to normal controls PB (44%) (p<0.01) (see FIG. 3). Although the T_{REG} cells were increased in the PB, they remained non-activated (CD86⁻) and generally naïve (CD45RA⁺) T cells (see FIG. 4). In the synovial tissue (ST) and interface tissue (IT), about 62% of the lymphocytes isolated were CD4⁺CD25⁺ T_{REG} cells (see FIG. 1). There was an increase in T cell activation (CD80⁺CD86⁺) in the ST and IT samples but not in the PB samples (CD80⁻CD86⁻) (see FIG. 5). The T_{REG} cells from the ST and IT samples were also memory like cells CD45RO⁺ (see FIG. 4). The presence of T_{REG} cells in the ST and IT were confirmed with immunohistochemistry.

Conclusion: Non-activated memory-like T_{REG} cells are upregulated in the peripheral blood of patients with failed total hips secondary to osteolysis/aseptic loosening. The T_{REG} cells are also present in the synovial tissue and interface tissue with an increase in T cell activation. Therefore, regulatory T cells are activated (CD80/86⁺) at the site of action of osteolysis. These T_{REG} cells appear to play a role in the pathogenesis of osteolysis in loose total hip replacements.

While the present invention has been described with reference to what are presently considered to be a preferred example, it is to be understood that the invention is not limited to the disclosed example. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of detecting osteolysis in a patient comprising:
(a) obtaining a sample containing lymphocytes from the patient; and
(b) determining the amount of CD4⁺CD25⁺ regulatory T cells wherein an increase in regulatory T cells as compared to a control indicates that the patient has osteolysis.

2. A method of detecting osteolysis according to claim 1 comprising:
(a) obtaining a sample containing lymphocytes from the patient; and
(b) determining the amount of CD4⁺CD25⁺ regulatory T cells as a percentage of the total CD4⁺ lymphocytes in the sample, wherein a level of regulatory T cells of greater than 45% indicates that the patient has osteolysis.

3. A method according to claim 2 wherein the regulatory T cells are present in an amount of greater than 50% of the total lymphocytes in the sample.

4. A method according to claim 2 wherein the regulatory T cells are present in an amount of greater than 60% of the total lymphocytes in the sample.

5. A method according to claim 2 wherein the regulator T cells are present in an amount of greater than 70% of the total lymphocytes in the sample.

6. A method according to claim 1 wherein the sample is peripheral blood.

7. A method according to claim 6 wherein the regulatory T cell has the phenotype CD3⁺CD4⁺CD25⁺CD45RA⁺.

8. A method according to claim 7 wherein the regulatory T cells are non-activated (CD80/86⁻).

9. A method according to claim 1 wherein the sample is synovial fluid, synovial tissue or interface tissue between the failed component and the bone defect.

10. A method according to claim 9 wherein the regulatory T cells are activated (CD80/86⁺).

11. A method according to claim 1 wherein the osteolysis is a result of total hip replacement.

12. A method according to claim 1 wherein the osteolysis is a result of primary or metastatic bone cancer.

13. A method according to claim 1 wherein the osteolysis is a result of a metabolic bone disease.

14. A method according to claim 13 wherein the metabolic bone disease is Paget disease.

15. A kit for detecting osteolysis comprising reagents for detecting CD4⁺CD25⁺ regulatory T cells as well as instructions for the use of the kit.

16. A kit according to claim 15 wherein the reagents comprise antibodies that bind to CD4 and antibodies that bind to CD25.
